(19) United States
(12) Patent Application Publication
(10) Pub. No.: US 2008/0228406 A1
(43) Pub. Date: Sep. 18, 2008

(54) SYSTEM AND METHOD FOR FUNGAL IDENTIFICATION

(75) Inventors: David W. DENNING, Cheshire (GB); Paul BOWYER, Cheshire (GB)

Correspondence Address: FOLEY AND LARDNER LLP;SUITE 500, 3000 K STREET NW, WASHINGTON, DC, 20007, US

(73) Assignee: Myconostica LTD.

(21) Appl. No.: 12/046,130

(22) Filed: Mar. 11, 2008

Related U.S. Application Data
(60) Provisional application No. 60/894,289, filed on Mar. 12, 2007.

(51) Int. Cl. G01N 33/48

(52) U.S. Cl. 702/19

(57) ABSTRACT

A service for the identification of at least one fungal organism suspected of being present in a test sample is described and comprises a processing capacity for the sample, a sequencing capacity for generating sequence information, a sequence information processing capacity, an assessment capacity, and a reporting capacity for reporting results to an end user. Also described are methods and systems of determining the presence and identity of at least one fungus in a sample.

### CROSS-REFERENCE TO RELATED PATENT APPLICATIONS

This application claims priority to U.S. Provisional Application 60/894,289 filed Mar. 12, 2007, which is incorporated herein by reference in its entirety.

### BACKGROUND

The following description is provided simply as an aide in understanding the invention and is not admitted to describe or constitute prior art to the invention.

Over 600 species of fungi produce clinical disease in man. Approximately twenty species cause greater than 98% of fungal infections. Common fungi can be known variously by species names such as, but not limited to, Candida, Aspergillus, Cryptococcus, various dermatophytes and some endemic fungi. Most of these are readily identified in clinical laboratories using a variety of phenotypic methods, particularly classical identification methods. The remaining 2% of fungal infections involve rarer fungi such as, but not limited to, Fusarium spp., Histoplasma, Coccidioides, Alternaria, etc. Medically important fungi and their identification are described by Larone. See Davise H. Larone, Medically Important Fungi: A Guide to Identification (4^{th} ed., 2002).

Classical identification systems work reasonably well for most fungi, although there are many examples of misidentification, sometimes with important clinical consequences. Training to properly identify fungal species can take years, however. Identification of rarer fungi is more problematic, as many laboratories are not equipped to make such determinations and must forward samples to a second laboratory. Such transfers to specialized laboratories can result in delays in treatment. Additional delays can arise from re-culturing the sample on different media or at different temperatures and from the performance of specialized tests. The delays can produce tragic outcomes for sufferers of fungal and/or bacterial infections.

In addition, for rarer fungi, identification is only one step in what can be a time consuming process to determine a treatment regimen. After identification, literature searches are normally needed to find agents that can treat the infection effectively. These literature searches are normally conducted by hand. Such searches are time-consuming and delay treatment, which can impact significantly a patient's recovery.

Therefore, a system is needed to provide clinicians with rapid, accurate analyses of a suspected fungal infection, along with up-to-date information regarding potential additional health hazards and appropriate remedial actions.

### SUMMARY

In one aspect, a service for the identification of at least one fungal organism suspected of being present in a test sample, which service does not rely exclusively on growing any fungal organism in culture to arrive at an identification is provided, comprises: (a) a mailroom capacity for providing one or more shipment kits, including one or more mailing labels, to an end-user for the shipment of one or more test samples comprising one or more unprocessed samples, processed samples, purified nucleic acids, one or more amplicons produced from purified nucleic acids, or combinations thereof; (b) an optional test sample processing capacity, including the capacity to purify nucleic acids from the one or more unprocessed samples, produce one or more amplicons from purified nucleic acids, or both; (c) a sequencing capacity for generating sequence information from one or more amplicons produced from purified nucleic acids; (d) a sequence information processing capacity, including the capacity for comparing the generated sequence information with sequence information stored in a database; (e) an assessment capacity, including the capacity to evaluate results from the comparison and implement a decision tree; (f) a reporting capacity for communicating one or more reports, including information: (i) identifying one or more fungal organisms, if any, likely present in the one or more test samples, (ii) summarizing potential clinical or environmental implications of the presence of the one or more fungal organisms identified, and (iii) recommending one or more remedial actions, if appropriate. In some embodiments, the service can be purchased online by the end user.

In some embodiments, the mailroom capacity also includes the capacity to receive the test sample from the end-user.

In some aspects, the one or more test samples are obtained from a biological source, including, but not limited to, a mammal susceptible to or suspected of suffering from a fungal infection, a tissue culture, or a cell culture. In some embodiments, the one or more test samples are obtained from a clinical specimen. In some such embodiments, the clinical specimen is a processed clinical specimen.

In other embodiments, the one or more test samples are obtained from a patient's environment. In such embodiments, the patient's environment includes a hospital room, a hospital water supply, the patient's home, the patient's workplace and the patient's automobile.

In some embodiments, the one or more mailing labels includes a unique barcode, while in others the one or more test samples are shipped to a recipient automatically designated by the service.

In some embodiments, the one or more amplicons includes at least one universal region of fungal nucleic acid, at least one region of fungal nucleic acid specific to a particular genus, at least one region of fungal nucleic acid specific to a particular species, or combinations thereof. In others, the one or more amplicons includes at least one region of fungal nucleic acid that confers antifungal drug resistance.

In some embodiments, the generated sequence information is uploaded into a computer server and/or saved in a storage medium.

In some embodiments, the comparison provides an output, including a plurality of possible matches associated with increasing or decreasing confidence levels.

In some embodiments, the assessment capacity includes a capacity to evaluate quality assurance issues, achievement of match criteria, potentially ambiguous results, merits of sequencing another target in an amplicon, or combinations thereof.

In some embodiments, the decision tree includes consideration of at least the following outcomes: no fungal organism found, no matches found, list of possible matches narrow or broad, no known pathological conditions associated with identified fungal organism, resistance to or ineffectiveness of selected antifungal agents, similarity of a given isolate to another for molecular typing purposes, population genetics data for a series of isolates and likelihood ratios of isolates arising from the same source.

In some embodiments, the assessment capacity includes input from a medical practitioner, infectious disease specialist, healthcare professional, taxonomist, mycologist, evolutionary biologist or other expert.

In some embodiments, the one or more reports are communicated via electronic mail. In some such embodiments, an email message is transmitted to the end-user notifying the availability of the one or more reports online. In other such embodiments, the email notification includes an access code, user name, or password to permit the end-user to access the one or more reports online.

In some embodiments, the one or more reports are made available within 72 hours of the end-user's purchase of the service. In other such embodiments, the one or more reports are made available within 48 hours of the end-user's purchase of the service. In yet other such embodiments, the one or more reports are made available within 5 or 7 working days of the end-user's purchase of the service. In some embodiments, an interim report is provided to the end-user. In another embodiment, partial identification data are provided and the end-user can elect to have additional work done for an additional payment. In one example, initial testing may reveal the genus of the fungus, but additional analysis may be required to identify the species. Alternatively, multiple fungi may be identified in a sample and additional testing may be required to ascertain resistance information for all the strains. In another example, the end-user, with the fungus identity in-hand, can request additional, specific analyses to assist in their epidemiological investigation.

In another aspect, a method for determining the presence and identity of at least one fungus in a clinical sample comprises (A) contacting nucleic acid from said sample with PCR reagents specific for at least one target fungal polynucleotide and amplicon the at least one target fungal polynucleotide to obtain at least one target fungal amplicon; (B) determining the sequence of said at least one target fungal amplicon; (C) identifying the species from which the at least one target fungal polynucleotide originated by comparing the sequence of said at least one target fungal amplicon to a set of known fungal nucleic acid sequences; and (D) formulating a report on potential clinical implications of the presence of the identified fungus and, if appropriate, on potential remedial actions. In some embodiments, the species identification is based on probability analyses or decision tree analyses.

In another aspect, a system for determining the presence and identity of at least one fungus in a clinical sample comprises (A) means for amplifying at least one target fungal polynucleotide present in said sample to obtain at least one target fungal amplicon; (B) means for determining the sequence of said at least one target fungal amplicon; (C) means for comparing the sequence of said at least one target fungal amplicon to a set of known fungal nucleic acid sequences; and (D) means for formulating a report on potential clinical implications of the presence of the identified fungus and, if appropriate, on potential remedial actions.

Other objects, features and advantages will become apparent from the following detailed description. The detailed description and specific examples are given for illustration only since various changes and modifications within the spirit and scope of the invention will become apparent to those skilled in the art from this detailed description.

### BRIEF DESCRIPTION OF THE DRAWINGS

FIG. 1 schematically depicts a process of elucidating the identity of fungi in a specimen using sequenced-based identification.

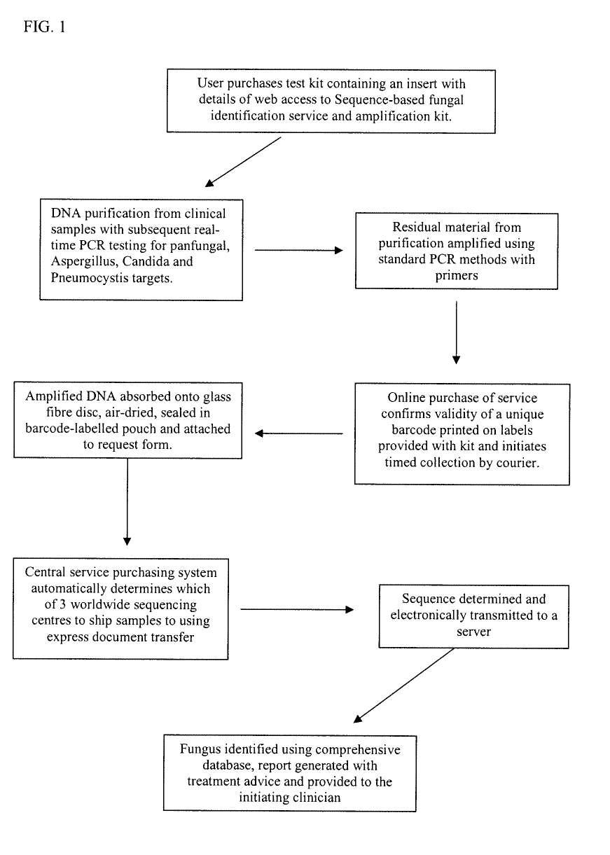

FIG. 2 illustrates sequential steps in the analysis of a sample, according to one embodiment.

FIG. 3 illustrates the order database architecture for the service, according to one embodiment.

FIG. 4 is an order database schema, according to one embodiment.

FIG. 5 is a sequence database schema, according to one embodiment.

FIG. 6 shows molecular typing schema and resistance database schema, according to one embodiment.

FIG. 7 is a knowledge database schema, according to one embodiment.

### DETAILED DESCRIPTION

In one aspect, services, methods and systems are provided for identifying at least one fungal organism suspected of being present in a test sample without relying on phenotypic identification. Instead, the services, methods and systems involve amplifying at least one target fungal polynucleotide from a sample, determining the sequence of the fungal amplicon, identifying the species from which the target fungal polynucleotide originated by comparing the sequence to known fungal nucleic acid sequences, and formulating a report on potential implications of the presence of the identified fungus and, if appropriate, on potential remedial actions. The disclosed services, methods and systems simplify the process of identifying fungi and dramatically shorten the time required for taking remedial action.

Unless indicated otherwise, all technical and scientific terms are used herein in a manner that conforms to common technical usage. Generally, the nomenclature of this description and the described laboratory procedures, including cell culture, molecular genetics, and nucleic acid chemistry and hybridization, respectively, are well known and commonly employed in the art. Standard techniques are used for recombinant nucleic acid methods, oligonucleotide synthesis, cell culture, tissue culture, transformation, transfection, transduction, analytical chemistry, organic synthetic chemistry, chemical syntheses, chemical analysis, and pharmaceutical formulation and delivery. Generally, enzymatic reactions and purification and/or isolation steps are performed according to the manufacturers' specifications. Absent an indication to the contrary, the techniques and procedures in question are performed according to conventional methodology disclosed. Specific scientific methods relevant to the present invention are discussed in more detail below. However, this discussion is provided as an example only, and does not limit the manner in which the methods of the invention can be carried out.

### DEFINITIONS

Unless otherwise specified “a” or “an” means one or more.

Unless otherwise specified “about” means to be within 10% of the stated value.

“Amplicon” refers to an exact replicate of the original template prepared by PCR techniques.

The term “end-user” refers to a person who obtains a test sample, provides treatment regimens, and/or is under the direction of a person obtaining a test sample or providing treatment regimens. In other embodiments, the term denotes the entity furnishing the test sample. Thus, an end-user can be a human or an entity, such as a clinic, hospital, laboratory which can provide a test sample to the service and to which the service furnishes a report.

“BAL” is an abbreviation for bronchioalveolar lavage. BAL is a medical procedure in which a bronchoscope is passed through the mouth or nose into the lungs and fluid is squirted into a small part of the lung and then recollected for examination.

“BP” is an abbreviation for base pair.

“ITS” is an abbreviation for internal transcribed spacer. The ITS is a sequence of RNA in a primary transcript that lies between precursor ribosomal subunits and is removed by splicing when the structural RNA precursor molecule is processed into a ribosome.

“Panfungal” refers to all or nearly all fungal species. In some embodiments, the term “panfungal” refers to all fungal species that can infect animals, including humans.

“PCR” is an abbreviation for polymerase chain reaction. PCR is a technique which is used to amplify the number of copies of a specific region of DNA, in order to produce enough DNA to be adequately tested.

“PCR reagents” refers to the chemicals, apart from the target nucleic acid sequence, needed to perform the PCR process. These chemicals generally consist of five classes of components: (i) an aqueous buffer, (ii) a water soluble magnesium salt, (iii) at least four deoxyribonucleotide triphosphates (dNTPs), (iv) oligonucleotide primers (normally two primers for each target sequence, the sequences defining the 5′ ends of the two complementary strands of the double-stranded target sequence), and (v) a polynucleotide polymerase, preferably a DNA polymerase, more preferably a thermostable DNA polymerase, i.e., a DNA polymerase which can tolerate temperatures between 90° C. and 100° C. for a total time of at least 10 minutes without losing more than about half its activity. The four conventional dNTPs are thymidine triphosphate (dTTP), deoxyadenosine triphosphate (dATP), deoxycytidine triphosphate (dCTP) and deoxyguanosine triphosphate (dGTP). These conventional triphosphates can be supplemented or replaced by dNTPs containing base analogues which Watson-Crick base pair like the conventional four bases, e.g., deoxyuridine triphosphate (dUTP).

“Polynucleotide” and “oligonucleotide” refer to polymers of nucleotide monomers, including analogs of such polymers, including double and single stranded deoxyribonucleotides, ribonucleotides, α-anomeric forms thereof, and the like. Descriptions of how to synthesize oligonucleotides can be found, among other places, in U.S. Pat. Nos. 4,373,071; 4,401,796; 4,415,732; 4,458,066; 4,500,707; 4,668,777; 4,973,679; 5,047,524; 5,132,418; 5,153,319; and 5,262,530. Polynucleotides and oligonucleotides can be of any length.

“SNP” is an abbreviation for single nucleotide polymorphism. A SNP is a small genetic change, or variation, that can occur within a person's DNA sequence.

In one aspect, a service for the identification of at least one fungal organism suspected of being present in a test sample comprises: (a) a mailroom capacity for providing one or more shipment kits, including one or more mailing labels, to an end-user for the shipment of one or more test samples comprising one or more unprocessed samples, processed samples, purified nucleic acids, one or more amplicons produced from purified nucleic acids, or combinations thereof; (b) an optional test sample processing capacity, including the capacity to purify nucleic acids from the one or more unprocessed samples, produce one or more amplicons from purified nucleic acids, or both; (c) a sequencing capacity for generating sequence information from one or more amplicons produced from purified nucleic acids; (d) a sequence information processing capacity, including the capacity for comparing the generated sequence information with sequence information stored in a database; (e) an assessment capacity, including the capacity to evaluate results from the comparison and implement a decision tree; (f) a reporting capacity for communicating one or more reports, including information: (i) identifying one or more fungal organisms, if any, likely present in the one or more test samples, (ii) summarizing potential clinical or environmental implications of the presence of the one or more fungal organisms identified, and (iii) recommending one or more remedial actions, if appropriate. In some embodiments, the service can be purchased online by the end user.

### Sample Acquisition

Samples can be obtained using a variety of methods known to those of skill in the art. For example, a sample can be obtained via any of a number of sources and/or methods including, but not limited to, blood, oral swabs, anal swabs, skin swabs, sputum, BAL, tissue samples, tissue cultures, food or condiment samples, food cultures, wipes, air or water filters or samplers, personal exposure nasal filters and other sources and/or methods of obtaining a sample known to those of skill in the art. Kits for collecting samples also are available.

In some embodiments, a test sample can be obtained from a biological source, such as a human subject or an animal subject. In some aspects, the subject is suspected of being infected by a fungal organism. In other embodiments, the test sample is obtained from the environment with which subject has had contact. Such environments can include the subject's home, pillows, bedding, clothes, automobile, workplace, hospital room, hospital, home water supply, hospital water supply, veterinary clinic, veterinary clinic water supply, showerheads, flowers and indoor plants and their container and soil or water, heating or air conditioning systems in the subject's home, hospital, hospital room, or veterinary clinic.

### Mailroom Capacity

The service comprises a mailroom capacity both for sending test kits and for receiving test samples from the clinician. In the former, a clinician, or end-user acting on behalf of a clinician, contacts that service for a test kit. Such contact can be via a telephone call, facsimile, email, or internet or website link, or via any other method known to those of skill in the art. The mailroom has the capacity to receive the request from the clinician and provide a package containing the test kit(s). The kit comprises, for example, directions for obtaining samples, using the test kit, and care and handling of the test kit, as well as packaging for an obtained sample. The package that is sent to the clinician also can contain return packaging, mailing labels, etc. The packaging, mailing labels, sample labels, etc. can contain information that is unique to each request for later, positive identification of the sample. The unique information can be recorded on the packaging, mailing label, and/or sample label as printed matter, a barcode, as magnetic data, or other identification means known to those of skill in the art.

In some aspects, test kits are provided to a clinician in advance of the suspicion of a fungal infection in a subject, thus allowing the clinician to have one or more test kits available at the clinician's locale. In such embodiments, the clinician then can obtain the sample, mail it to the service, and notify the service that the sample has been sent.

The mailroom also has the capacity to receive samples from a clinician. In some aspects, the sample is contained within a test kit provided by the service. Such capacity can include receipt of the sample, proper labeling and handling of the sample to ensure that the sample is properly correlated to the clinician from which the sample was sent, and transfer of the sample to an appropriate station within the service. Such capacity can operate on an around-the-clock basis, handling samples at any time of the day or night.

The mailroom also can have the capacity to send and receive invoices for the service to clients, end-users, clinicians, etc.

### Sample Processing

The samples obtained by the end-user can be raw and sent to the service for full processing and testing, or such samples can be processed by the clinician, or end-user. For example, with regard to the former, the end-user can provide to the service the sample as obtained from either the subject or the subject's environment. Alternatively, the end-user can process the sample as a fixed or unfixed histological specimen of tissue, a plasma specimen, a serum specimen, a whole blood sample, a sterile fluid such as cerebrospinal fluid, urine, vitreous fluid, BAL, or a wet preparation specimen from any body site, etc.

Fixed histological specimens include samples of biological tissue or fluid taken from a subject and treated with an agent to prevent decomposition of the tissue or fluid. Such methods of fixing specimens are well known to those of skill in the art.

Plasma specimens are those specimens in which plasma has been separated from whole blood by methods known to those of skill in the art. For example, gel formulations can be added to the whole blood to serve as a density separator, the sample then being centrifuged to separate the plasma from the whole blood.

Serum specimens are those specimens in which the plasma has been further separated from fibrin and other soluble clotting elements, but still contain the fungal organism, or DNA of the fungal organism.

Whole blood collected with an anticoagulant such as EDTA, and from which DNA is extracted using one a variety of methods which might include red cell hypotonic lysis, enzymatic or physical treatments to disrupt fungal or other cells, and precipitation techniques to concentrate the DNA.

Wet preparation specimens are those specimens of biological origin that are liquid that can be fixed with an agent to prevent degradation of the sample. The preparation and handling of wet preparation specimens are known to those of skill in the art.

Techniques for processing samples are well known to those of skill in the art. Such sample processing can include various steps to separate target nucleic acids from other biological material. Examples include, standard ethanol precipitation, as well as commercial preparations, such as those from available from Qiagen Inc. (Valencia, Calif.).

In still other embodiments, a sample is partially processed by a clinician and/or the end-user and finalized by the service. This can include further processing of an unprocessed sample, production of one or more amplicons using purified nucleic acids, or both. Such further processing can include subjecting the test sample to a sequencing regimen, as a step toward identification of the fungal organism.

In some embodiments, the test sample processing capacity includes the capacity to generate information on restriction length fragment polymorphisms (RLFP) from purified nucleic acids or the one or more amplicons produced from purified nucleic acids. RLFP is the identification of specific restrictions enzymes (REs) that reveal a pattern difference between the DNA fragment sizes in individual organisms. REs are DNA-cutting enzymes found in bacteria (and harvested from them for use). A RE recognizes and cuts DNA only at a particular sequence of nucleotides. To discover RFLPs, REs are used to cut DNA at specific 4-6 BP recognition sites. Sample DNA is cut with one or more REs and the resulting fragments are separated according to molecular size using gel electrophoresis. Differences in fragment length result from base substitutions, additions, deletions, or sequence rearrangements within RE recognition sequences.

In other embodiments, the test sample processing capacity includes the capacity to generate information on multiple SNPs from purified nucleic acids or the one or more amplicons produced from purified nucleic acids, as part of a scheme to generate a molecular signature through multilocus sequence typing (MLST). MLST is a procedure for characterizing isolates of bacterial species using the sequences of internal fragments of (usually) seven house-keeping genes.

In MLST, approximately 450-500 BP internal fragments of each gene are used, as these can be accurately sequenced on both strands using an automated DNA sequencer. For each house-keeping gene, the different sequences present within a fungal species are assigned as distinct alleles and, for each isolate, the alleles at each of the seven loci define the allelic profile or sequence type (ST). Each isolate of a species is therefore unambiguously characterized by a series of seven integers which correspond to the alleles at the seven house-keeping loci. In MLST the number of nucleotide differences between alleles is ignored and sequences are given different allele numbers whether they differ at a single nucleotide site or at many sites. The rationale is that a single genetic event resulting in a new allele can occur by a point mutation (altering only a single nucleotide site), or by a recombinational replacement (that will often change multiple sites)—weighting according to the number of nucleotide differences between alleles would erroneously consider the latter allele to be more different to the original allele than the latter. See M. C. Maiden et al., Proc. Natl. Acad. Sci. USA, 95, 3140-3145 (1998) and R. Urwin et al., Trends Microbiol., 11, 479-487 (2003).

In yet other embodiments, the test sample processing capacity includes the capacity to generate information on microsatellite lengths from purified nucleic acids or the one or more amplicons produced from purified nucleic acids, as part of a scheme to generate a molecular signature or multi-locus microsatellite typing (MLMT) system. MLMT is a procedure similar to MLST using microsatellite loci for positive identification.

### Amplification of Target Nucleic Acids

In one aspect, at least one target nucleic acid in a sample is amplified. A variety of techniques can be utilized to amplify target nucleic acids. Amplification reactions typically use isolated, purified or recombinant nucleic acid enzymes to replicate specific nucleic acids. Depending on the amplification reaction, nucleic acid enzymes can have template-dependent nucleic acid polymerase activity, RNA polymerase activity, DNA polymerase activity or reverse transcriptase activity. Both mesophilic and thermophilic enzymes can be used. Exemplary amplification reactions include, but are not limited to, a polymerase chain reaction, a ligase chain reaction, a loop-mediated isothermal amplification, a nucleic acid sequence based amplification, a self-sustained sequence replication, a strand displacement amplification, and a transcription mediated amplification.

In one embodiment, a target nucleic acid can be amplified by a polymerase chain reaction (“PCR”). PCR is a well known amplification reaction for amplifying specific nucleic acid segments. PCR amplifies specific DNA segments by cycles of template denaturation; primer addition; primer annealing and replication using thermostable DNA polymerase. Exemplary protocols for PCR can be found, for example, in U.S. Pat. Nos. 4,683,195 and 4,683,202, which are hereby incorporated by reference. A thermostable nucleic acid polymerase is relatively stable to heat when compared, for example, to nucleotide polymerases from E. coli which catalyze the polymerization of nucleoside triphosphates. Generally, the enzyme initiates synthesis at the 3′-end of the primer annealed to the target nucleic acid, and will proceed in the 5′-direction along the target nucleic acid, and if possessing a 5′-to-3′ nuclease activity, hydrolyzing intervening, annealed probe to release both labeled and unlabeled probe fragments, until synthesis terminates. A representative thermostable enzyme isolated from Thermus aquaticus (Taq) is described in U.S. Pat. No. 4,889,818 and a method for using it in conventional PCR is described in Saiki et al., 1988, Science 239:487. Taq DNA polymerase has a DNA synthesis-dependent, strand replacement 5′-3′ exonuclease activity (see Gelfand, “Taq DNA polymerase” in PCR Technology: Principles and Applications for DNA Amplification, Erlich, Ed., Stockton Press, N.Y. (1989)). PCR can be coupled with the use of a nucleic acid enzyme having reverse transcriptase activity to amplify RNA.

A wide variety of instrumentation has been developed for carrying out PCR. Examples can be found in Johnson et al, U.S. Pat. No. 5,038,852 (computer-controlled thermal cycler); Woudenberg et al., U.S. Pat. No. 6,015,674, Wittwer et al, Nucleic Acids Research, 17: 4353-4357 (1989)(capillary tube PCR); Hallsby, U.S. Pat. No. 5,187,084 (air-based temperature control); Garner et al, Biotechniques, 14: 112-115 (1993) (high-throughput PCR in 864-well plates); Wilding et al, International application No. PCT/US93/04039 (PCR in micro-machined structures); and Schnipelsky et al, European patent application No. 90301061.9 (publ. No. 0381501 A2) (disposable, single use PCR device), all of which are hereby incorporated by reference.

In other embodiments, the target nucleic acid can be amplified by ligase chain reaction (“LCR”). LCR is an nucleic acid amplification reaction similar to PCR. LCR differs from PCR as the oligonucleotide probe is the template of the amplicon as opposed to the target nucleic acid. In LCR, two oligonucleotide probes are used per each strand of nucleic acid. The probes are designed to exactly match two adjacent sequences of a specific target DNA. LCR uses both a DNA polymerase enzyme and a DNA ligase enzyme to drive the reaction. Like PCR, LCR requires a thermal cycler to drive the reaction and each cycle results in a doubling of the target nucleic acid molecule. The chain reaction is repeated in three steps in the presence of excess probe: (1) heat denaturation of double-stranded DNA, (2) annealing of probes to target DNA, and (3) joining of the probes by thermostable DNA ligase. Exemplary protocols for LCR are found, for example, in Landegren et al., Science 241: 1077-1080 (1988); D. Y. Wu and R. B. Wallace, Genomics 4:560-569 (1989); and F. Barany, PCR Methods Appl. 1:5-16 (1991), which are hereby incorporated by reference. LCR can be coupled with the use of a nucleic acid enzyme having reverse transcriptase activity to amplify RNA.

In another embodiment, the target nucleic acid can be amplified by loop-mediated isothermal amplification (“LAMP”). LAMP is an amplification reaction technique with high specificity, efficiency and rapidity under isothermal conditions. In LAMP, a DNA polymerase and four specially designed primers recognize a total of six distinct sequences on a target nucleic acid. An inner primer containing sequences of the sense and antisense strands of the target nucleic acid initiates LAMP. Strand displacement by nucleic acid synthesis primed by an outer primer releases a single-stranded nucleic acid. This single-stand nucleic acid acts as template for nucleic acid synthesis primed by a second set of inner and outer primers. These primers hybridize to the other end of the target, thereby producing a stem-loop DNA structure. In subsequent LAMP cycling, one inner primer hybridizes to the loop on the product and initiates displacement DNA synthesis, yielding the original stem-loop DNA and a new stem-loop DNA with a stem twice as long. Exemplary protocols for LAMP amplification reactions are found, for instance, in Nagamin et al., Clin. Chem. 47(9):1742-1743 (2001); Notomi et al., Nucleic Acids Res. 28(12):E63 (2000), which are hereby incorporated by reference. Because it is an isothermal reaction, mesophilic enzymes having nucleic acid polymerase activity can be used to drive the amplification reaction. For example, the DNA polymerase large fragment (Klenow fragment) from E. coli is suitable for use in LAMP.

In another embodiment, the target nucleic acid can be amplified by nucleic acid sequence based amplification (“NASBA”). NASBA is a primer-dependent amplification reaction technique used for the isothermic amplification of nucleic acids. NASBA amplification can be performed on both RNA and DNA target nucleic acids. For RNA, NASBA is initiated by the annealing of an oligonucleotide primer to the RNA target nucleic acid. The 3′ end of the primer is designed such that it is complementary to the target nucleic acid and, at the 5′ end, encodes the T7 RNA polymerase promoter. After annealing, the reverse transcriptase activity of AMV-RT is engaged and a cDNA copy of the RNA target is produced. The RNA portion of the resulting hybrid molecule is hydrolyzed through the action of RNase H. Once sufficiently complete, a second primer, which is complementary to an upstream portion of the RNA target nucleic acid, anneals to the cDNA strand. The DNA-dependent DNA polymerase activity of AMV-RT is engaged again, thereby producing a double stranded cDNA copy of the original RNA target nucleic acid with a fully functional T7 RNA polymerase promoter at one end. NASBA next utilizes T7 RNA polymerase to produce a large amount of anti-sense, single stranded RNA transcripts corresponding to the original RNA target. These anti-sense RNA transcripts can serve as templates for the amplification process, however the primers anneal in the reverse order. For DNA, the process is the same except that an initial heat denaturing step is required before the addition of the enzymes to the reaction mix. An exemplary protocol for NASBA is found in J. Compton, Nature 350:91-92 (1991), which is hereby incorporated by reference.

In yet another embodiment, the target nucleic acid can be amplified by self-sustained sequence replication (“3SR”). 3SR is an isothermal amplification reaction which utilizes three enzymatic activities essential to retroviral replication: reverse transcriptase, RNase H, and a DNA-dependent RNA polymerase. Generally, 3SR mimics the retroviral strategy of RNA replication by means of cDNA intermediates, As such, 3SR accumulates cDNA and RNA copies of the original target nucleic acid. In 3SR, the amplicon accumulates exponentially with respect to time, indicating that newly synthesized cDNAs and RNAs function as templates for a continuous series of transcription and reverse transcription reactions. An exemplary protocol for 3SR, including exemplary enzymes, is found in Guatelli et al., Proc. Natl. Acad. Sci. U.S.A. 87(5): 1874-1878 (1990), which is hereby incorporated by reference.

In another embodiment, the target nucleic acid can be amplified by strand displacement amplification (“SDA”). SDA is an isothermal amplification reaction technique based upon the ability of a restriction enzyme to nick the unmodified strand of a hem-modified DNA recognition site and the ability of a 5′-3′ exonuclease deficient DNA polymerase to extend the 3′ end at the nick and displace the downstream strand. SDA achieves exponential target nucleic acid amplification by coupling sense and antisense reactions in which strands displaced in a sense reaction serve as target nucleic acids for an antisense reaction and vice versa. Exemplary protocols for SDA are found, for example, in Walker et al., Nucleic Acids Res., 20:1691-1696 (1992); and Walker et al., Proc. Natl. Acad. Sci. U.S.A. 89:392-396 (1992), which are hereby incorporated by reference.

The target nucleic acid can also be amplified by transcription mediated amplification (“TMA”). TMA is an amplification reaction technique which utilizes a nucleic acid enzyme having RNA transcription activity and a second nucleic acid enzyme having DNA synthesis activity (i.e. reverse transcriptase) to produce an RNA amplicon from a target nucleic acid. TMA can be used to target both RNA and DNA. An exemplary TMA protocol is found, for example, in Pasternack et al., J. Clin. Microbiol. 35(3):676-678 (1997), which is hereby incorporated by reference.

Apparatuses for carrying out such amplifications are well-known in the art and are specifically disclosed in the references above. In addition, those skilled in the art will recognize that other amplification reaction methodologies and techniques can be used.

Amplicons can be detected using a variety of indicator molecules. An “indicator molecule” is any molecule which can be used to determine the presence of amplification product during or after an amplification reaction. The skilled artisan will appreciate that many indicator molecules can be used in the present invention. For example, according to certain embodiments, indicator molecules include, but are not limited to, fluorophores, radioisotopes, chromogens, enzymes, antigens, heavy metals, dyes, magnetic probes, phosphorescence groups, chemiluminescent groups, and electrochemical detection moieties.

A “fluorescent indicator” is any molecule or group of molecules designed to indicate the amount of amplification product by a fluorescent signal. In certain embodiments, such fluorescent indicators are “nucleic acid binding molecules” that bind or interact, e.g., through ionic bonds, hydrophobic interactions, or covalent interactions with nucleic acid. Complex formation with the minor groove of double stranded DNA, nucleic acid hybridization, and intercalation are all non-limiting examples of nucleic acid binding for the purposes of this invention. In certain embodiments, such fluorescent indicators are molecules that interact with double stranded nucleic acid. In certain embodiments, fluorescent indicators can be “intercalating fluorescent dyes,” which are molecules which exhibit enhanced fluorescence when they intercalate with double stranded nucleic acid. In certain embodiments, “minor groove binding fluorescent dyes” can bind to the minor groove of double stranded DNA. In certain embodiments, fluorescent dyes and other fluorescent molecules can be excited to fluoresce by specific wavelengths of light, and then fluoresce in another wavelength. According to certain embodiments, dyes can include, but are not limited to, acridine orange; ethidium bromide; thiazole orange; pico green; chromomycin A3; SYBR® Green I (see U.S. Pat. No. 5,436,134); quinolinium, 4-[(3-methyl-2(3H)-benzoxazolylidene) methyl]-1-[3-(trimethylammonio) propyl]-, diiodide (YOPRO®); and quinolinium, 4-[(3-methyl-2(3H)-benzothiazolylidene) methyl]-1-[3-(trimethylammonio) propyl]-, diiodide (TOPRO®). SYBR® Green I, YOPRO®, and TOPRO® are available from Molecular Probes, Inc., Eugene, Oreg.

According to certain embodiments, the fluorescent indicators can be 5′-nuclease fluorescent indicators, which are fluorescent molecules attached to fluorescence quenching molecules by a short oligonucleotide. According to certain embodiments, the fluorescent indicator binds to the target molecule, but is broken by the 5′ nuclease activity of the DNA polymerase when it is replaced by the newly polymerized strand during PCR, or some other strand displacement protocol. When the oligonucleotide portion is broken, the fluorescent molecule is no longer quenched by the quenching molecule, and emits a fluorescent signal. An example of such a 5′-nuclease fluorescent indicator system has been described in U.S. Pat. No. 5,538,848, and is exemplified by the TaqMan™ molecule, which is part of the TaqMan™ assay system (available from Applied Biosystems).

According to certain embodiments, the fluorescent indicators can be “molecular beacons,” which comprise a fluorescent molecule attached to a fluorescence-quenching molecules by an oligonucleotide. When bound to a polynucleotide as double stranded nucleic acid, the quenching molecule is spaced apart from the fluorescent molecule, and the fluorescent indicator can give a fluorescent signal. When the molecular beacon is single stranded, the oligonucleotide portion can bend flexibly, and the fluorescence-quenching molecule can quench the fluorescent molecule, reducing the amount of fluorescent signal. Such systems are described in U.S. Pat. No. 6,150,097, which is hereby incorporated by reference.

During amplification, an indicator molecule is included in the amplification reaction. According to certain embodiments, this molecule indicates the amount of double-stranded DNA in the reaction, and thus serves as a measure of the amount of amplification product produced. In certain embodiments, the indicator molecule is a fluorescent indicator. In certain embodiments, the fluorescent indicator is a nucleic acid binding molecule which binds with the DNA, resulting in a change in its fluorescent qualities. Exemplary dyes of this type include, but are not limited to, acridine orange, ethidium bromide, and SYBR® Green I (Molecular Probes, Inc.) (see U.S. Pat. No. 5,436,134).

In certain embodiments, the fluorescent indicator can be a fluorescing dye connected to a quenching molecule by a specific oligonucleotide. These include, but are not limited to, 5′-nuclease fluorescent indicators and molecular beacons. Examples of such systems are described, e.g., in U.S. Pat. Nos. 5,538,848 and 5,723,591.

### Sequencing Capacity

In one aspect, the sequence of at least one target nucleic acid in a sample is determined. In some embodiments, sequence information is obtained for more than one gene. In one example, regions of the genes for 18S, internal transcribed spacer (ITS) regions and calmodulin are sequenced. In some embodiments, genes associated with drug resistance also are sequenced. In other embodiments, MLST is undertaken, as a secondary step.

The sequence of a target nucleic acid can be determined by a variety of methods known in the art. In one aspect, the sequence is elucidated via cycle sequencing, which is described generally in U.S. Pat. Nos. 5,821,058; 5,332,666 and 5,171,534, all of which are hereby incorporated by reference. A wide variety of instrumentation has been developed for performing cycle sequencing. Examples include genetic analyzers from Applied Biosystems, including the 3100 series of Genetic Analyzers, such as the 3130 DNA Analyzer, and the 3730 DNA Analyzer, and the 3730x1 DNA Analyzer. Other such devices comprising a thermal cycler, light beam emitter, and a fluorescent signal detector, such as the ABI Prism® 5700, also are available from Applied Biosystems, and have been described in U.S. Pat. Nos. 5,928,907; 6,015,674; and 6,174,670, all of which are hereby incorporated by reference.

High-throughput sequencing technologies also can be employed. Neil Hall, Journal of Experimental Biology, 209:1518-1525 (2007); G. M. Church, Scientific American, 294 (1): 47-54 (2006). For example, emulsion PCR involves isolating individual DNA molecules along with primer-coated beads in aqueous bubbles within an oil phase. A PCR then coats each bead with clonal copies of the isolated library molecule, and these beads are subsequently immobilized for later sequencing. Commercial applications are available from 454 Life Sciences and Applied Biosystems' SOLID™ System. See Margulies, et al, Nature 437: 376-380 (2005);

Shendure et al., Science 309 (5741): 1728-1732, all of which are hereby incorporated by reference.

Another method for in vitro clonal amplification is “bridge PCR”, where fragments are amplified upon primers attached to a solid surface. These methods both produce many physically isolated locations which each contain many copies of a single fragment. Commercial kits are available from Illumina.

Another high-throughput method involves parallelized sequencing, where once clonal DNA sequences are physically localized to separate positions on a surface, various sequencing approaches may be used to determine the DNA sequences of all locations, in parallel. In one example “sequencing by synthesis”, uses the process of DNA synthesis by DNA polymerase to identify the bases present in the complementary DNA molecule. Reversible terminator methods use reversible versions of dye-terminators, adding one nucleotide at a time, detecting fluorescence corresponding to that position, then removing the blocking group to allow the polymerization of another nucleotide. Pyrosequencing also uses DNA polymerization to add nucleotides, adding one type of nucleotide at a time, then detecting and quantifying the number of nucleotides added to a given location through the light emitted by the release of attached pyrophosphates. Ronaghi et al., Analytical Biochemistry 242: 84-89 (1996).

“Sequencing by ligation” is another enzymatic method of sequencing, using a DNA ligase enzyme rather than polymerase to identify the target sequence. See U.S. Pat. No. 5,750,341, which is hereby incorporated by reference. This method uses a pool of all possible oligonucleotides of a fixed length, labeled according to the sequenced position. Oligonucleotides are annealed and ligated. The preferential ligation by DNA ligase for matching sequences results in a signal corresponding to the complementary sequence at that position.

In another example, “sequencing by hybridization” can be used. Such methods typically involve the use of a DNA microarray. Typically, a single pool of unknown DNA is fluorescently labeled and hybridized to an array of known sequences. If the unknown DNA hybridizes strongly to a given spot on the array, causing it to “light up”, then that sequence is inferred to exist within the unknown DNA being sequenced. See, e.g. G. J. Hanna et al., Journal of Clinical Microbiology, 38 (7): 2715 (2000), which is hereby incorporated by reference.

Mass spectrometry also can be used to sequence DNA molecules. Conventional chain-termination reactions produce DNA molecules of different lengths, and the length of these fragments is then determined by the mass differences between them (rather than using gel separation).

In certain embodiments, combined thermal cycling and fluorescence detecting devices can be used for precise quantification of target polynucleotides in samples. In certain embodiments, fluorescent signals can be detected and displayed during or after each thermal cycle, thus permitting monitoring of amplification products as the reactions occur in “real time.” In certain embodiments, one can use the amount of amplification product and number of amplification cycles to calculate how much of the target polynucleotide was in the sample prior to amplification.

According to certain embodiments, one can simply monitor the amount of amplicon(s) after a predetermined number of cycles sufficient to indicate the presence of the target polynucleotide in the sample. The number of cycles sufficient to determine the presence of a given target polynucleotide for any given sample type, primer sequence or reaction condition is readily determinable by those skilled in the art.

In certain embodiments, the results of the amplification reaction are used to determine which of the amplification products to subject to sequencing reactions. In certain embodiments, the sequencing reactions are then read by a sequencing apparatus to determine the sequence. In some aspects, the sequencing apparatus is automatic.

In some embodiments, the one or more amplicons includes at least one universal region of fungal nucleic acid, at least one region of fungal nucleic acid specific to a particular genus, at least one region of fungal nucleic acid specific to a particular species, or a combination of any two or more thereof. In other embodiments, the one or more amplicons includes at least one region of fungal nucleic acid that confers antifungal drug resistance. By exploiting such regions of fungal nucleic acid, the process of identification and treatment with antifungal agents, described below is much more facile.

In some embodiments, the identifier region is the ITS region. Thus, a kit can provide sequencing primers for the PCR/sequencing, with subsequent sequence information provided locally or under contract with a large rapid sequencing company.

### Identification of the Isolate

Once the sequence of the target fungal nucleic acid has been elucidated, the identify of the isolate can be determined by comparing the target sequence to known fungal sequences in, for example, a database. The fungi in the database with the most homologous nucleic acid sequence is considered the most closely related species to the fungus in the test sample. In this regard, the target fungal nucleic acid can have 100% homology to a sequence in the database. In other embodiments, the target fungal nucleic acid is identified when there is less than 100% homology with a sequence in the database, such as 99%, 98%, 97%, 96% or 95%. Such lower levels of homology may allow consideration of the isolate identity to be from the same genus, but a different species. If such target fungal sequences have less homology a different species and genus is likely, and may thus be considered to be a new species. Interpretation of the precise percentage homology scores that confer different species or genus vary with the sequenced region and phylogeny of the reference species.

In other embodiments, sequence identity can be used to compare the target fungal sequence with known sequences. As used herein, “percentage of sequence identity” means the value determined by comparing two optimally aligned sequences over a comparison window, wherein the portion of the polynucleotide sequence in the comparison window can comprise additions or deletions (i.e., gaps) as compared to the reference sequence (which does not comprise additions or deletions) for optimal alignment of the two sequences. The percentage of sequence identity is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

In one aspect, at least one database is utilized that comprises a phylogenetic classification of fungi based upon nucleic acid sequences. The database can contain a variety of nucleic acids from a variety of fungal genes. In some embodiments, the database includes sequences for ribosomal RNA (rRNA). In this regard, the database can comprise, for example, 16S rRNA, 18S rRNA, and 26S rRNA or the ITS regions.

The database can be searched using a computer. In one aspect, the database is accessed locally, whereas in another the database is accessed via the internet. In other aspects, a combination of a variety of databases can be searched, comprising public and private databases. Examples of public DNA databases, include GenBank, EMBL and DDBJ, as well as Ribosomal Database Project installed in the University of Michigan. In addition, the Fungal Tree of Life (AFTOL) project may be referenced and searched. The AFTOL attempts to describe approximately 1.5 million species of fungi. The AFTOL consists of a framework (WASABI—Web Accessible Sequence Analysis for Biological Inference) that includes continuously updated databases that are accessible via the Internet. WASABI facilitates collection and dissemination of molecular data to and from laboratories and participants. WASABI allows data to be viewed, downloaded and verified. The WASABI interface includes basecalling of newly generated chromatograms, contig assembly, quality verification of sequences (including BLAST), sequence alignment and congruence test. Verified gene sequences will undergo automated phylogenetic analysis on a regular schedule.

A variety of alignment tools can be used to compare the sequences. Optimal alignment of sequences for comparison can be conducted by the local homology algorithm of Smith and Waterman, Adv. Appl. Math. 2: 482 (1981); by the homology alignment algorithm of Needleman and Wunsch, J. Mol. Biol. 48: 443 (1970); by the search for similarity method of Pearson and Lipman, Proc. Natl. Acad. Sci. 85: 2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; and the CLUSTAL program described in Higgins and Sharp, Gene 73: 237-244 (1988); Higgins and Sharp, CABIOS 5: 151-153 (1989); Corpet, et al., Nucleic Acids Research 16: 10881-90 (1988); Huang, et al., Computer Applications in the Biosciences 8: 155-65 (1992), and Pearson, et al., Methods in Molecular Biology 24: 307-331 (1994).

The BLAST family of programs which can be used for database similarity searches includes: BLASTN for nucleotide query sequences against nucleotide database sequences; BLASTX for nucleotide query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995); Altschul et al., J. Mol. Biol., 215:403-410 (1990); and, Altschul et al., Nucleic Acids Res. 25:3389-3402 (1997).

Software for performing BLAST analyses is publicly available, e.g., through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults, a wordlength (W) of 11, an expectation (E) of 10, a cutoff of 100, M=5, N=−4, and a comparison of both strands (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5877 (1993)). One measure of similarity provided by the BLAST algorithm is the smallest sum probability (P(N)), which provides an indication of the probability by which a match between two nucleotide or amino acid sequences would occur by chance.

BLAST searches assume that proteins and nucleotides can be modeled as random sequences. However, many proteins and nucleotides comprise regions of nonrandom sequences which can be homopolymeric tracts, short-period repeats, or at least for the case of proteins, regions enriched in one or more amino acids. Such low-complexity regions can be aligned between unrelated proteins even though other regions of the protein are entirely dissimilar. A number of low-complexity filter programs can be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters can be employed alone or in combination.

Multiple alignment of the sequences can be performed using the CLUSTAL method of alignment (Higgins and Sharp (1989) CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

Many fungal ITS sequences are available in public databases. For example, the MicroSeq 16S rDNA Sequence Database (trademark, of PE Biosystems Japan, Ltd.), and commercially available software, such as MicroSeq Analysis Software (trademark of PE Biosystems Japan, Ltd.) are readily available. However, several of pathogenic fungi are very under-represented, and in some cases non-existent, leading to either no positive identification or a suspect positive identification that requires further testing. In such cases, traditional testing methods can be used to positively identify the fungal organism, with the sequencing information then added to the database, thus expanding the database for future identifications. In other such cases, professional input from a person of skill in the art can be used to assist in the identification of the fungal organism when no positive identification or a suspect positive identification results. Such persons can include, a medical practitioner, infectious disease specialist, hospital epidemiologist, healthcare professional, taxonomist, mycologist, evolutionary biologist, or other expert.

In other aspects, the service provides sequencing information processing to compare the generated sequences to sequence information stored in a database to provide identification of the suspect fungal and/or bacterial organism. In some instances such information will be integral to the sequencing capacity instrument. In other instances, independent databases can be assembled or used to identify sequences that are typical of a given fungal and/or bacterial organism. In some embodiments, this involves uploading the generated sequence information to a computer server and/or saved in a storage medium. While in other embodiments, the comparison provides an output, including a plurality of possible matches associated with increasing or decreasing confidence levels. The assessment capacity can include a capacity to evaluate quality assurance issues, achievement of match criteria, potentially ambiguous results, merits of sequencing another target in an amplicon, or a combination of any two or more thereof.

In some embodiments, the service provides an assessment capacity, including the capacity to evaluate results from the comparison and implement a decision tree. Such a capacity can be computer driven or can require further information or decisions by an operator to proceed. For example, the decision tree can include consideration of at least the following outcomes: no fungal and/or bacterial organism found, no matches found, a list of possible matches (narrow or broad), no known pathological conditions associated with identified fungal and/or bacterial organism, resistance to or ineffectiveness of selected antifungal and/or antibacterial agents, similarity of a given isolate to another for molecular typing purposes, population genetics data for a series of isolates, and likelihood ratios of isolates arising from the same source.

In assessing the identity of the suspect fungal organism, or isolate, a number of factors can be taken into consideration. Such as the symptoms presented by the subject from which the sample was obtained, the subject's medical and personal history, the subject's geographical origin, the results of other radiological or diagnostic tests, the results of the sequencing and other such factors that can be known to those of skill in the art.

The decision tree can include, in some embodiments, input from persons of skill in the art. Such persons can include, a medical practitioner, infectious disease specialist, healthcare professional, taxonomist, mycologist, evolutionary biologist, or other expert. Such input can be useful in cases where the sequencing analysis identifies a rare fungus. Such input also can be beneficial in cases where sequencing analysis reveals multiple species present in a sample. In such cases, an expert using the subject's history, sample origin, geographical area, etc. can identify the various species. Expert input also can be helpful in determining appropriate remedial actions, if any. Such actions can include administering antifungal treatments, for example a pharmaceutical. Another action, such as building remedial works, could result from cluster analysis of related or identical fungi from common environments and infected patients. In some instances, remedial action is unnecessary. For example, the subject's own immune system can be allowed to fight the fungal infection, or the fungal infection can be allowed to run it's course. In some embodiments, experts can direct and oversee the system and apparatuses to ensure generation of an accurate report.

### The Report

In one aspect, a report is formulated that describes the potential implications of the presence of the identified fungus and, if appropriate, potential remedial actions. In one example, a report is generated that identifies the species and/or genus of the organism, known potential clinical or environmental implications of such an organism, possible remedial actions, if any, known antifungal treatments such as appropriate pharmaceutical intervention, a glossary of technical terms used in the report for the convenience of a non-expert clinician, and other information concerning the identified fungal organism. Thus, the generated report rapidly provides a clinician with sufficient information to prescribe specific treatment.

With regard to reporting the identification of the species, a number of outcomes are possible, as alluded to above. For example, a single fungal species can be present, in which case positive identification is a likely outcome of the analysis and the positive identification can be reported to the end-user. However, in other instances, a multitude of fungal species may be present, as evidenced by a mixed population of sequences. As one of skill in the art will readily appreciate, positive identification of the species present in a test sample containing a number of species presents difficulties in positive identification. In such instances, an positive answer for any single species may not be possible, and thus the report may contain such information. In some cases, sequencing may show that one species dominates over another, and may also show genus-level similarities. Thus, the report can state that a number of fungal species are present, and if known, the genus to which the species belong, as even genus information can be relevant to potential remedial actions.

In some embodiments, the report will provide one or more of the following: the closest fungal identification possible, a prepared text on that species describing its approximate frequency as a cause of disease, the type of infection, allergic syndromes it is known to produce, the patient groups with which it is most often associated, geographical associations (such as the endemic mycoses (histoplasmosis, coccidioidomycosis etc), serology or other non-culture means to confirm the diagnosis, and intrinsic and acquired antifungal resistance characteristics associated with the species. A direct link to a Medline/Pubmed search also can be provided.

In other embodiments, the report also can provide information detailing previous observations regarding sensitivity or resistance of the identified organisms to antifungal agents. Where the fungal species cannot be unambiguously identified, information regarding sensitivity or resistance of the closest known relative to the identified organisms to antifungal agents may be supplied. Expert observations can be furnished in the report, based on previously recorded instances of infection with the identified organism. Such observations can include, but are not limited to, the suitability of a particular antifungal agent(s), potential route(s) of administration, the length of treatment, the level or dosage of the antifungal agent to be used, and/or an assessment of the likely virulence of the identified organism based on past case history.

A different report form would be provided to deliver the result of MLST or other molecular typing, for example. Not only would the report detail the genus and species (if determined) of the fungus, but it also would disclose its molecular type. This report also would provide information on other molecular types of this fungus, and a subsidiary cumulative report from single institutions would be sent to the end-user encompassing all isolates of that species from that institution.

To determine possible remedial action, databases and published literature comprising information regarding antifungal agents can be searched, for example, by computer. Such databases should contain information on effective concentrations of various pharmaceutical agents for treating a variety of fungal infections. Such databases also can identify possible complications arising from the suggested treatment and/or suspected infection. The databases can be searched, for example, by fungal name or accession number.

Reports generated by the service can be provided to the end user by conventional mail, telephone, facsimile, via an internet webpage, or via electronic mail. Any of the methods of transmission of a report to the end user and/or clinician can include access code, user name, or password to permit the end-user to access the one or more reports electronically.

Databases of all the information compiled can be made to be accessible online an end-user, with accessibility provided through a username/password combination for a limited number of searches. This feature can then be optionally available to an end-user through the purchase of the test kit.

The service also will provide the information in a timely fashion. In some embodiments, the report is furnished to the end-user within 72 hours of the initial purchase, 60 hours of the initial purchase, within 48 hours of the initial purchase, within 36 hours of the initial purchase, or within 24 yours of the initial purchase. In some embodiments, the report is furnished to the end-user between about 12 hours and about 72 hours from the initial purchase; between about 24 hours and about 72 hours from the initial purchase; between about 36 hours and about 72 hours from the initial purchase; between about 12 hours and about 48 hours from the initial purchase; or between about 24 hours and about 48 hours from the initial purchase. In some embodiments, the initial purchase is defined as the time the end-user purchases a test kit online and the test kit is sent to the end-user. In other embodiments, the initial purchase is defined as the time the end-user uses a pre-purchased test kit and notifies the service that the test kit is being sent for analysis. In another embodiment, partial identification data are provided and the end-user can elect to have additional work done for an additional payment. In one example, initial testing may reveal the genus of the fungus, but additional analysis may be required to identify the species. Alternatively, multiple fungi may be identified in a sample and additional testing may be required to ascertain resistance information for all the strains. In another example, the end-user, with the fungus identity in-hand, can request additional, specific analyses to assist in their epidemiological investigation.

In some instances of rare fungi, or for fungi that present challenges in analysis, the length of time until a final report is furnished to the end-user may be from 5 to 14 days, or longer. In such instances, an interim report may be furnished to the end-user providing the information available during various stages of testing. Thus, in some embodiments, the report is furnished to the end-user from about 5 to about 14 days; from about 5 to about 12 days; from about 5 to about 10 days; or from about 5 to about 7 days from the initial purchase. In some such embodiments, an interim report may be issued.

In other embodiments, a test kit is provided for the detection of infection by any fungus. Besides kits for panfungal assays, test kits specific to a particular fungal species, or range of fungal species are provided. For example, the service can provide test kits to an end-user that are specific to Aspergillus/Candidal Pneumocystis species. In some cases, a panfungal assay can be positive, while the species specific test or assay can be negative, thus, indicating the presence of a rarer species of fungus. Thus, in some embodiments, the test kit includes PCR reagents for amplifying and then sequencing part of the genome, as described above.

### EXAMPLE 1

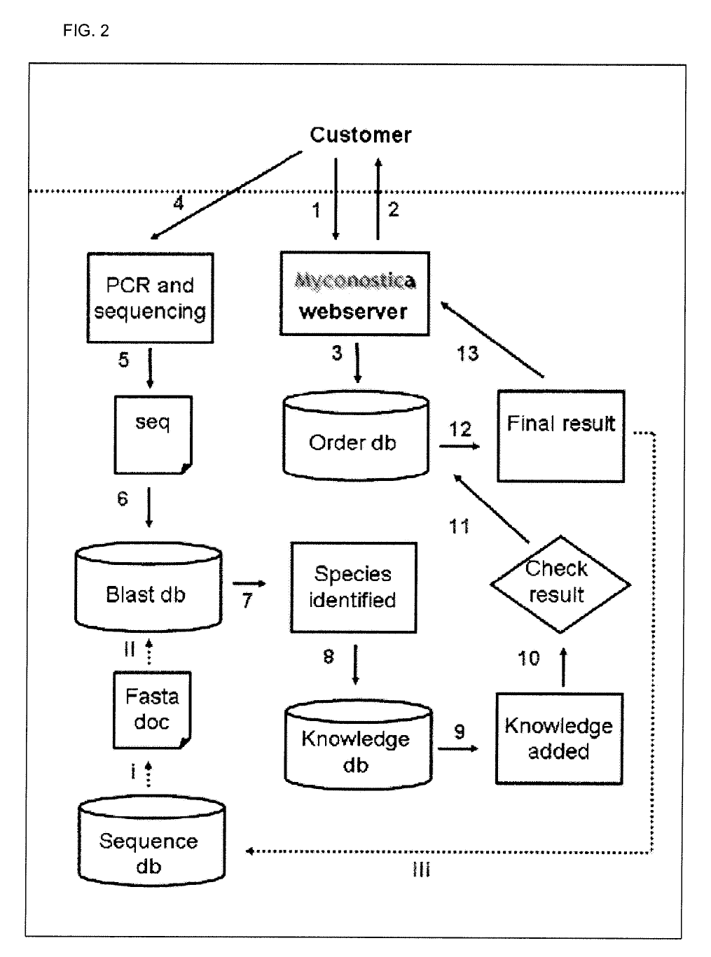

FIG. 2 illustrates sequential steps in the analysis of a sample, according to one embodiment. As shown in the Figure, dashed arrows indicate “off line” steps for the updating of the Sequence and Blast databases. First, a user accesses the website to submit an order (Step 1). A username and password are required. These will also enable the user to access a webpage with details of their results. Logging in allows the user to access a webpage where they will enter details of the sample they want analyzed.

Upon submitting their order, the user is provided with an ID number for their sample (sample identifier) (Step 2). The identifier is then sent with the order and will be used to track the sample during subsequent steps. It is useful to have a mechanism that checks the validity of the identifier. One possibility is to have a second number which is a product of the first. An example is credit card numbers which use a checksum to ensure the credit card number is valid. In some instances, several samples may be submitted at the same time. In this situation, a second identifier (order identifier) can be used. The details that the user submits will be stored in the order database. To simplify data entry, drop down menus are used whenever possible.

Identifiers and details about the user (e.g. name and email address) and about the sample (e.g. blood, pus, etc.) are stored in the order database (Step 3). Another identifier can chosen by the user, to help them link the resulting analysis with a patient (e.g. Patient id 1234XY22).

Next, the biological sample arrives (Step 4). The user is sent an email to confirm that the sample has been received. Alternatively, the user can track the delivery using standard tracking technology. Then, PCR amplification and DNA sequencing are carried out.

The sequence output is converted to FASTA format and edited to remove the primer sequences (Step 5). If more than one PCR product will be sequenced, multiple unique names will be generated from the sample identifier (e.g. Q2365_ITS1, Q2356-ITS2). The DNA sequence is blasted against the Blast database (Step 6). The Blast analysis identifies a best hit, which includes a fungal species identifier in the output (Step 7). The knowledge database is searched using the species identifier from the Blast hit (Step 8).

The customer database is searched using the sample identifier of the Blast input sequence (Step 9). The result of this search, plus the search of the knowledge database are merged to produce an analysis result document which contains details of the sequencing plus alignment of sample sequences from the Blast database, results from the knowledge database and details about the user.

The analysis result is checked to ensure that the species identification is reliable (Step 10). The result is emailed to the user (Step 11). The result is added to the Order database (Step 12). A PDF document (or html) giving details of the analysis result is created and placed in a password protected folder. The directory structure for the result documents is based upon the order and sample identifiers. The email contains a link to the result document (Step 13). The user name and password required to access the documents are the same as those that were used to log in and send the orders.

Regarding the “off line” steps, a Fasta file is generated from sequences in the sequence database (Step i). This file is indexed to generate the Blast database (Step ii). Sequences generated by the service can be added to the sequence database (Step iii).

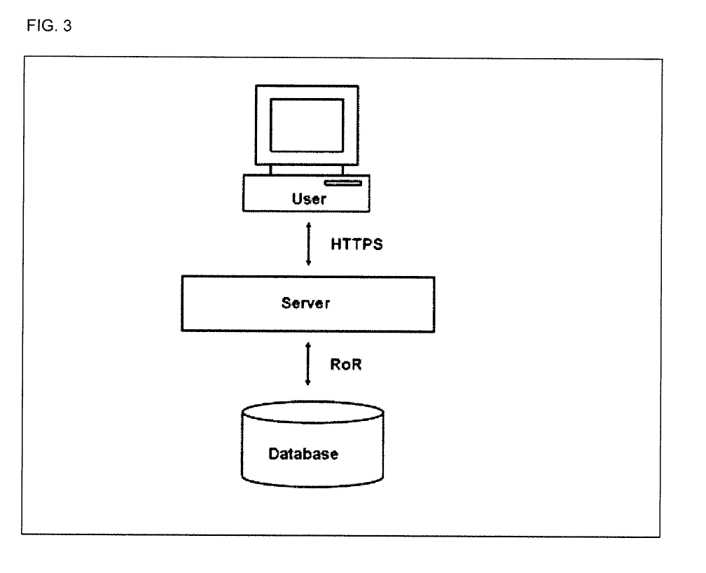

FIG. 3 illustrates the order database architecture for the service. The order database is the only part of the service infrastructure for which there is public access. This infrastructure is similar to other websites for e-Commerce and, thus, routine practices in the field can be used to set up the server and database.

Because the order database is visible to the public, security is very important. After log-in, all communication between the user and server is via HTTPS. This is essentially secure HTTP, meaning that information passed from the user to the web server is encrypted. In addition, a firewall can be used and preferably one that installs updates regularly. Anti-malware and spyware software also is used.

There are several methods by which the servers communicate and practitioners can utilize the method best suited for their particular application. One example is Ruby on Rails, which provides a framework for developing database backed web applications and works with a variety of web servers and databases.

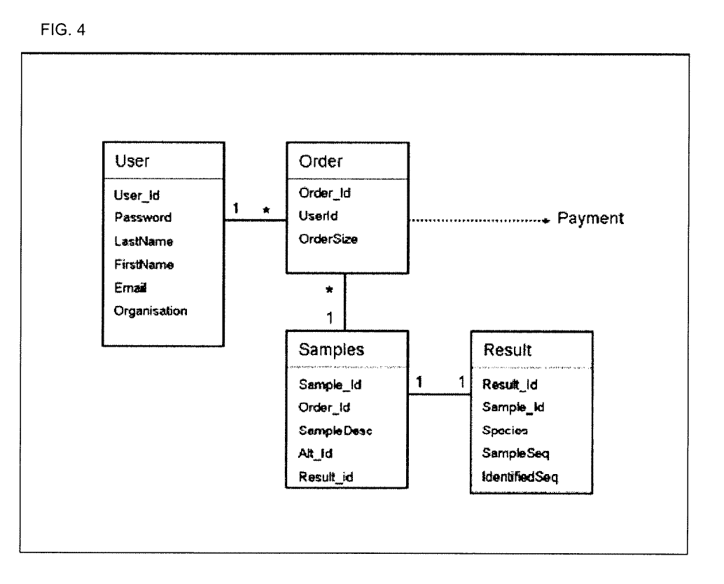

One possible schema for the Order database is shown in FIG. 4. As shown, a user can create many orders, an order can contain many samples and a sample will have a result (i.e. the identified fungal species). The orders also can contain data that will allow the service provider to keep track of payment for services.

The Blast database is different from the other databases discussed here in the sense that there is a no database management system. Instead, the sequences in the sequence database are output in Fasta format and this file is indexed to produce the Blast database.

The sequence database is initially populated using publicly available data from repositories such as Genbank. Possible analysis that can be carried out to assess the quality of sequence data are: All vs. all Blast analysis, Constructing a phylogenetic tree based upon the sequences and reclassification of sequences based on new information.

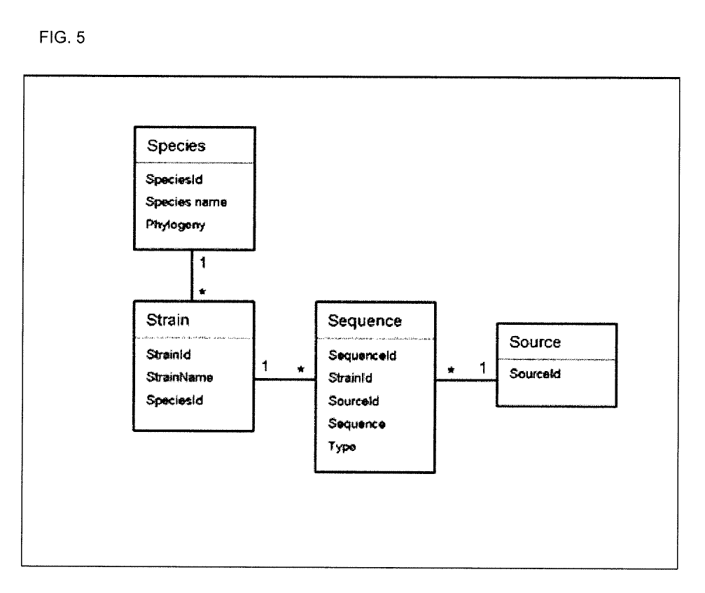

One possible schema for the sequence database is shown in FIG. 5. The schema allows for a species to have multiple strains, which may be advantageous if there are strain differences in terms of pathogenicity.

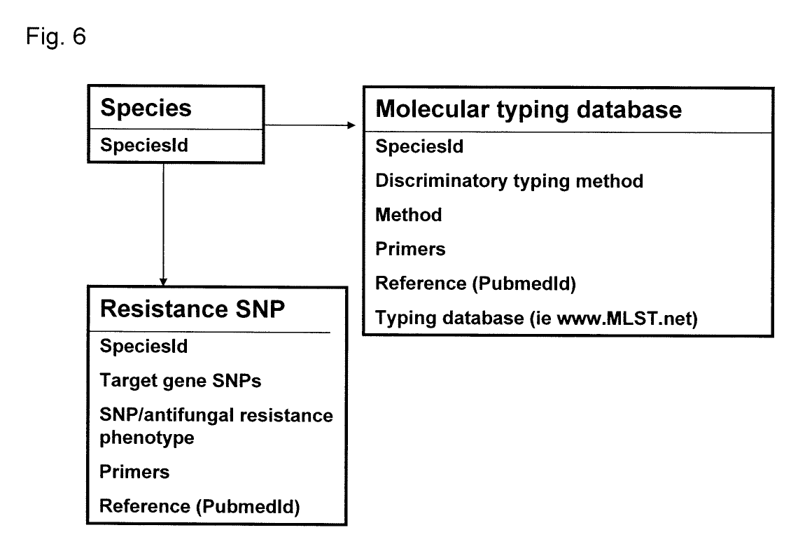

In some embodiments, following species identification, molecular typing and resistance databases may be used. FIG. 6 provides exemplary schema for such databases, which are used to identify the molecular typing or resistance information of certain fungi. A user can request such analyses, if available, when initially placing an order or after receiving notification of the availability of such analyses.

The knowledge database contains input from experts on different fungal species, including information on classification, drug resistance and effective methods of treatment and handling. Since contributors update the species database directly, contributors will have a username and password and will be limited in the alterations they can make. In some instances it might be appropriate to return knowledge on a closely related species.

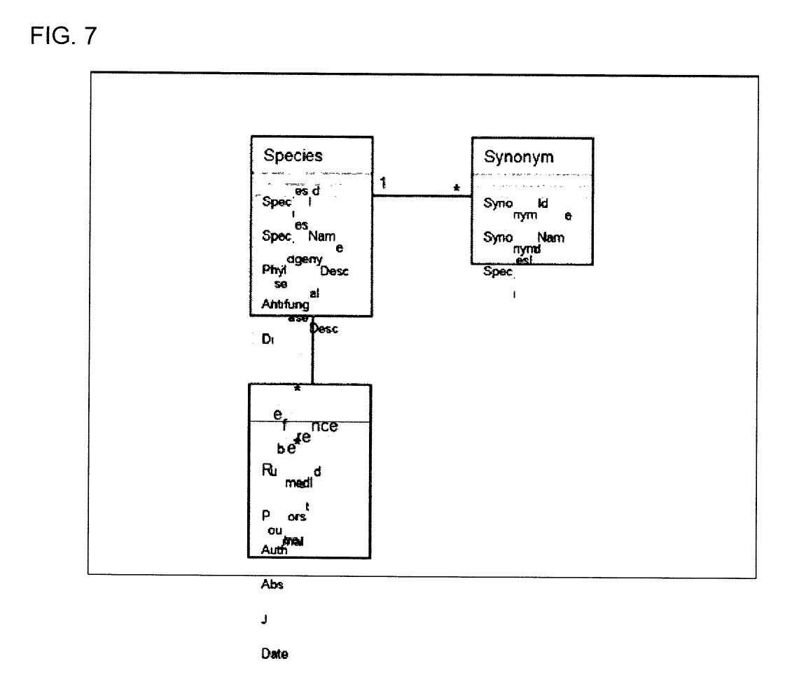

One knowledge database schema is shown in FIG. 7. A main function of the knowledge database is the generation of a report after a species has been associated with a sample.

Software takes a DNA sequence, compares it against the Blast database and then retrieves details from the order and knowledge databases. According to one embodiment the software is written using Perl scripts to run SQL which can be run from a command line. In order to minimize repetitions, sequences are placed in a folder and the script run over all sequences in the folder.

The largest files associated with this analysis are the sequence trace files. A typical sequence ab1 file has a size of 0.3 MB.

The hardware chosen should be resilient. This might include deploying a server with two CPUs. This will provide a back up should one of the processors fail. In addition, mirrored internal discs can be used. The system is installed simultaneously on the two internal hard disks, and all data is written to both disks. Further, an overnight backup to an external disc drive can be implemented. Alternatively, sequential changes can be stored to tape on a nightly basis and then a full back up to tape carried out weekly. Software is available to automate this process. Preferably, all three databases use relational database management system (RDBMS) software. These databases can be implemented using, for example, MySQL or Oracle.

While the invention is described with reference to exemplary embodiments, it will be understood by those skilled in the art that various changes can be made and equivalents can be substituted for elements thereof without departing from the scope of the invention. In addition, many modifications can be made to adapt a particular situation or material to the teachings of the invention without departing from the essential scope thereof. Therefore, it is intended that the invention not be limited to the particular embodiment disclosed as the best mode contemplated for carrying out this invention.

We claim:

1. A service for the identification of at least one fungal organism suspected of being present in a test sample, which service does not rely exclusively on growing any fungal organism in culture to arrive at an identification, the service comprising:
(a) a mailroom capacity for providing one or more shipment kits, including one or more mailing labels, to an end-user for the shipment of one or more test samples comprising one or more unprocessed samples, processed samples, purified nucleic acids, one or more amplicons produced from purified nucleic acids, or combinations thereof;
(b) an optional test sample processing capacity, including the capacity to purify nucleic acids from the one or more unprocessed samples, produce one or more amplicons from purified nucleic acids, or both;
(c) a sequencing capacity for generating sequence information from one or more amplicons produced from purified nucleic acids;
(d) a sequence information processing capacity, including the capacity for comparing the generated sequence information with sequence information stored in a database;
(e) an assessment capacity, including the capacity to evaluate results from the comparison and implement a decision tree;
(f) a reporting capacity for communicating one or more reports, including information: (i) identifying one or more fungal organisms, if any, likely present in the one or more test samples, (ii) summarizing potential clinical or environmental implications of the presence of the one or more fungal organisms identified, and (iii) recommending one or more remedial actions, if appropriate.

2. The service of claim 1 in which the one or more test samples is obtained from a biological source.

3. The service of claim 2 in which the biological source includes a mammal susceptible to or suspected of suffering from a fungal infection, a tissue culture or a cell culture.

4. The service of claim 1 in which the one or more test samples is obtained from a clinical specimen.

5. The service of claim 1 in which the one or more test samples is obtained from a processed clinical specimen.

6. The service of claim 1 in which the one or more test samples is obtained from a patient's environment.

7. The service of claim 6 in which the patient's environment comprises the group consisting of a hospital room, a hospital water supply, the patient's home, the patient's workplace and the patient's automobile.

8. The service of claim 1 which the end-user can purchase online.

9. The service of claim 1 in which the one or more mailing labels includes a unique barcode.

10. The service of claim 1 in which the one or more test samples are shipped to a recipient automatically designated by the service.

11. The service of claim 1 in which the one or more amplicons includes at least one universal region of fungal nucleic acid, at least one region of fungal nucleic acid specific to a particular genus, at least one region of fungal nucleic acid specific to a particular species, or combinations thereof.

12. The service of claim 1 in which the one or more amplicons includes at least one region of fungal nucleic acid that confers antifungal drug resistance.

13. The service of claim 1 in which the test sample processing capacity includes the capacity to generate information on restriction length fragment polymorphism (RLFP) from purified nucleic acids or the one or more amplicons produced from purified nucleic acids.

14. The service of claim 1 in which the test sample processing capacity includes the capacity to generate information on multiple single nucleotide polymorphisms (SNPs) from purified nucleic acids or the one or more amplicons produced from purified nucleic acids, as part of a scheme to generate a molecular signature, so called multilocus sequence typing (MLST).

15. The service of claim 1 in which the test sample processing capacity includes the capacity to generate information on microsatellite lengths from purified nucleic acids or the one or more amplicons produced from purified nucleic acids, as part of a scheme to generate a molecular signature, a multi-locus microsatellite typing (MLMT) system.

16. The service of claim 1 in which the generated sequence information is uploaded into a computer server and/or saved in a storage medium.

17. The service of claim 1 in which the comparison provides an output, including a plurality of possible matches associated with increasing or decreasing confidence levels.

18. The service of claim 1 in which the assessment capacity includes a capacity to evaluate quality assurance issues, achievement of match criteria, potentially ambiguous results, merits of sequencing another target in an amplicon, or combinations thereof.

19. The service of claim 1 in which the decision tree includes consideration of at least the following outcomes: no fungal organism found, no matches found, list of possible matches narrow or broad, no known pathological conditions associated with identified fungal organism, resistance to or ineffectiveness of selected antifungal agents, similarity of a given isolate to another for molecular typing purposes, population genetics data for a series of isolates and likelihood ratios of isolates arising from the same source.

20. The service of claim 1 in which the assessment capacity includes input from a medical practitioner, infectious disease specialist, hospital epidemiologist, healthcare professional, mycologist, taxonomist, evolutionary biologist or other expert.

21. The service of claim 1 in which the one or more reports include a glossary of technical terms used in the one or more reports.

22. The service of claim 1 in which the one or more reports are communicated via electronic mail.

23. The service of claim 1 in which an email message is transmitted to the end-user notifying the availability of the one or more reports online.

24. The service of claim 21 in which the email notification includes an access code, user name, or password to permit the end-user to access the one or more reports online.

25. The service of claim 1 in which the one or more reports are made available within 72 hours of the end-user's purchase of the service.

26. The service of claim 1 in which the one or more reports are made available within 48 hours of the end-user's purchase of the service.

27. The service of claim 1 in which the one or more reports are made available within 5 to 14 days of the end-user's purchase of the service.

28. The service of claim 1 in which the one or more remedial actions include a listing of pharmaceutical antifungal agents known to treat the fungal organism infecting a subject.

29. The service of claim 1 wherein the mailroom capacity further comprises the capacity for receiving the test sample from the end-user.

30. A method for determining the presence and identity of at least one fungus in a clinical sample, comprising
(A) contacting nucleic acid from said sample with PCR reagents specific for at least one target fungal polynucleotide and amplifying the at least one target fungal polynucleotide to obtain at least one target fungal amplicon;
(B) determining the sequence of said at least one target fungal amplicon;
(C) identifying the species from which the at least one target fungal polynucleotide originated by comparing the sequence of said at least one target fungal amplicon to a set of known fungal nucleic acid sequences; and
(D) formulating a report on potential clinical implications of the presence of the identified fungus and, if appropriate, on potential remedial actions.

31. The method of claim 30, wherein said species identification is based on probability analyses or decision tree analyses.

32. The method of claim 31, wherein the decision tree includes consideration of at least the following outcomes: no fungal organism found, no matches found, list of possible matches narrow or broad, no known pathological conditions associated with identified fungal organism, resistance to or ineffectiveness of selected antifungal agents, similarity of a given isolate to another for molecular typing purposes, population genetics data for a series of isolates and likelihood ratios of isolates arising from the same source.

33. The method of claim 30, wherein said report formulation is based on probability analyses or decision tree analyses.

34. A system for determining the presence and identity of at least one fungus in a clinical sample, comprising
(A) means for amplifying at least one target fungal polynucleotide present in said sample to obtain at least one target fungal amplicon;
(B) means for determining the sequence of said at least one target fungal amplicon;
(C) means for comparing the sequence of said at least one target fungal amplicon to a set of known fungal nucleic acid sequences; and
(D) means for formulating a report on potential clinical implications of the presence of the identified fungus and, if appropriate, on potential remedial actions.

35. The system of claim 34, wherein said species identification is based on probability analyses or decision tree analyses.

36. The system of claim 35, wherein the decision tree includes consideration of at least the following outcomes: no fungal organism found, no matches found, list of possible matches narrow or broad, no known pathological conditions associated with identified fungal organism, resistance to or ineffectiveness of selected antifungal agents, similarity of a given isolate to another for molecular typing purposes, population genetics data for a series of isolates and likelihood ratios of isolates arising from the same source.

37. A method of identifying a course of treatment for a patient suspected of having a fungal infection, comprising
(A) obtaining a sample from said patient
(B) determining a sequence of any fungal polynucleotide in said sample;
(C) identifying the species from which the fungal polynucleotide originated by comparing the fungal polynucleotide sequence to a set of known fungal nucleic acid sequences; and
(D) formulating a report on potential clinical implications of the presence of the identified fungus and, if appropriate, on potential remedial actions.

38. The method of claim 37, wherein said sequence determination comprises sequencing at least one region known to confer drug resistance.

39. The method of claim 38, wherein said region comprises the gene encoding AzRF1 transcription factor and a deletion of residues QSQS at position 553-556 of said mutant gene confers triazole-resistance.

40. The method of claim 37, wherein said sequence determination and report formulation are based on probability analyses or decision tree analyses
